(19) United States
(12) Patent Application Publication
(10) Pub. No.: US 2023/0206292 A1
(43) Pub. Date: Jun. 29, 2023

(54) IDENTIFYING HIGH COST IMAGING PROCEDURES HAVING SCOPE FOR OPERATIONAL AND FINANCIAL IMPROVEMENT

(71) Applicant: KONINKLIJKE PHILIPS N.V., EINDHOVEN (NL)

(72) Inventors: RANJITH NAVEEN TELLIS, TEWKSBURY, MA (US); USHANANDINI RAGHAVAN, LEXINGTON, NY (US); THUSITHA DANANJAYA DE SILVA MABOTUWANA, REDMOND, WA (US); YUECHEN QIAN, LEXINGTON, MA (US)

(21) Appl. No.: 17/913,203

(22) PCT Filed: Mar. 17, 2021
(86) PCT No.: PCT/EP2021/056838

§ 371 Date: Sep. 21, 2022

Related U.S. Application Data
(60) Provisional application No. 62/994,341, filed on Mar. 25, 2020.

(51) Int. Cl. G06Q 30/04; G06Q 10/04; G16H 40/20

(52) U.S. Cl. CPC G06Q 30/04; G06Q 10/04; G16H 40/20

(57) ABSTRACT

A non-transitory computer readable medium (26) stores instructions readable and executable by at least one electronic processor (20) to perform medical procedure billing rate optimization method (100). The method includes: fitting a parametric probability density function (PDF) (38) to a dataset of turnaround times (TATs) for a medical procedure; drawing a number of occurrences of the medical procedure from the dataset of TATs or the fitted parametric PDF; generating a present billing rate (40) for the medical procedure based on an amount billed per procedure for the drawn occurrences of the medical procedure, and the TATs for the drawn occurrences of the medical procedure; drawing a number of hypothetical occurrences of the medical procedure from the fitted parametric PDF with at least one parameter modified to represent a hypothetical change in the medical procedure; generating a hypothetical billing rate (42) for the medical procedure with the hypothetical change based on a hypothetical amount billed per procedure for the drawn hypothetical occurrences of the medical procedure, and the TATs for the drawn hypothetical occurrences of the medical procedure; and providing a user interface (UI) (28) displaying a visualization (44) of a comparison of the present billing rate and the hypothetical billing rate for the medical procedure with the hypothetical change.

### FIELD

The following relates generally to the medical procedure auditing arts, workflow efficiency analysis and improvement arts, financial data modeling arts, statistical modeling arts, what-if scenario generation, and related arts.

### BACKGROUND

Radiology departments and other medical laboratories are constantly under pressure to deliver high quality patient care with a smaller annual budget, caused by challenges posed by regulatory and reimbursement model changes, amongst other factors. The volume and complexity of medical imaging studies continue to rise. With the advent of Accountable Care Organizations (ACOs), medical service departments are driven to continue to demonstrate the clinical and financial utility of their services. One of the ways to improve quality is to focus on improving the operational efficiency and reducing wasted time for those medical procedures that have greater impact on the financial returns.

By way of illustration, a radiology workflow typically consists of performing imaging procedures using various modalities like X-rays, Magnetic Resonance Imaging (MRI), Computed Tomography (CT), Ultrasound (US), among others. Radiology departments continuously monitor and adjust workflow by focusing on the timeline or turn-around-time (TAT) of imaging studies starting from order-to-schedule, schedule-to-appointment, appointment-to-patient arrival, arrival-to-begin of exam, begin-to-end of exam and then to final read and communication of diagnostic findings.

In particular, an “exam TAT” is defined herein as when a technologist begins the first scan on the patient and ends when the technologist declares the images as ready for a radiologist to read. Exam TATs are important to the overall workflow in a radiology department and multiple stakeholders influence the performance. Operations managers monitor exam TATs to ensure they have sufficient capacity to manage the number of patients to be scanned on a specific day. Radiologists heavily influence exam TATs by prescribing scanning protocols that can provide optimal images to support them in diagnostic findings. The technology and technologists working with patients also influence the time spent (and variations in time) in performing the imaging exams.

There exists a need to identify procedures that have scope for operational improvement and has the potential to generate higher financial returns when optimized compared to the rest of the procedures performed at the imaging center.

The following discloses certain improvements to overcome these problems and others.

### SUMMARY

In one aspect, a non-transitory computer readable medium stores instructions readable and executable by at least one electronic processor to perform medical procedure billing rate optimization method. The method includes: fitting a parametric probability density function (PDF) to a dataset of turnaround times (TATs) for a medical procedure; drawing a number of occurrences of the medical procedure from the dataset of TATs or the fitted parametric PDF; generating a present billing rate for the medical procedure based on an amount billed per procedure for the drawn occurrences of the medical procedure, and the TATs for the drawn occurrences of the medical procedure; drawing a number of hypothetical occurrences of the medical procedure from the fitted parametric PDF with at least one parameter modified to represent a hypothetical change in the medical procedure; generating a hypothetical billing rate for the medical procedure with the hypothetical change based on a hypothetical amount billed per procedure for the drawn hypothetical occurrences of the medical procedure, and the TATs for the drawn hypothetical occurrences of the medical procedure; and providing a user interface (UI) displaying a visualization of a comparison of the present billing rate and the hypothetical billing rate for the medical procedure with the hypothetical change.

In another aspect, an apparatus includes a display device; and at least one electronic processor programmed to: generate a present billing rate for a medical procedure based on a number of occurrences of the medical procedure, an amount billed for the determined number of occurrences of the medical procedure, and a turn-around-time for the number of occurrences of the medical procedure; generate a hypothetical billing rate based with at least one parameter modified to represent a hypothetical change in the medical procedure; and provide a user interface on the display device displaying a visualization of a comparison between the present billing rate and the hypothetical billing rate.

In another aspect, a medical procedure billing rate optimization method includes: fitting a PDF to a dataset of TATs for a medical procedure; generating a present billing rate for the medical procedure based on an amount billed per procedure for occurrences of the medical procedure drawn from the dataset of TATs for the medical procedure, and the TATs for the drawn occurrences of the medical procedure; generating a hypothetical billing rate for the medical procedure with the hypothetical change based on a hypothetical amount billed per procedure for hypothetical occurrences of the medical procedure drawn from the fitted parametric PDF with at least one parameter modified to represent a hypothetical change in the medical procedure, and the TATs for the drawn hypothetical occurrences of the medical procedure; providing a UI displaying a visualization of a comparison of the present billing rate and the hypothetical billing rate for the medical procedure with the hypothetical change; generating different hypothetical billing rates for the fitted parametric PDF with different modifications of at least one parameter of the fitted parametric PDF representing corresponding different hypothetical changes in the medical procedure; and updating the visualization of the comparison of the present billing rate and the different hypothetical billing rates corresponding to the different hypothetical changes in the medical procedure.

One advantage resides in providing a system for use in determining an optimized billing rate for a medical procedure.

Another advantage resides in providing a system for use in determining an optimized billing rate by quantitatively assessing the financial impact of reducing TATs of medical procedures.

Another advantage resides in providing a system for analyzing performance of a medical facility for the purpose of increasing revenue for a medical facility performing medical procedures.

Another advantage resides in providing a system for analyzing performance of a medical facility for the purpose of decreasing TATs for medical procedures in a cost-efficient way.

Another advantage resides in providing a system for performing what-if scenarios to generate optimized billing rates for medical procedures, thereby increasing revenue for a medical facility where the medical procedure is performed.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for analyzing medical procedure billing rates in accordance with the present disclosure.

FIG. 2 shows an example output by the apparatus of FIG. 1.

FIG. 3 shows an example dialog screen displayed on the apparatus of FIG. 1.

### DETAILED DESCRIPTION

The following relates to systems and methods for performing analysis of turnaround time (TAT) statistics for medical imaging procedures, with the goal of identifying procedures which can be improved in order to improve profitability. Identifying procedures that have large variability in their exam times provides a good starting point for identifying procedures that can benefit from being optimized for efficiency. But that alone is not sufficient in identifying which set of procedures when optimized has greater impact on the financial returns for the hospital.

Illustrative examples described herein relate to optimizing medical imaging procedures. An imaging procedure refers to a certain type of imaging scan performed on a patient using a certain modality for example an “X-Ray chest 1 view”. The hospital revenue is represented by the billing amount charged for the procedure. This can very well be the actual reimbursed amount received by the insurance company or patient (i.e., the payment amount), net profits, or any other value that needs to be maximized. Typically, a medical imaging procedure is billed at a fixed billing amount, regardless of how long it takes to perform the procedure. For example, medical insurance companies typically assign International Classification of Diseases (ICD) codes to medical procedures and reimburse a given ICD code at a fixed reimbursement (i.e., billing) amount. Also as previously mentioned, an “exam TAT” is defined as when a technologist begins the first scan on the patient and ends when the technologist declares the images as ready for a radiologist to read. While the billing amount can often be modeled as a constant value per instance of the procedure, the scan TAT can vary widely between imaging technicians and specific patients. However, the billing amount can also vary. For example, the billing amount can be different based on a contract between the insurance companies, and therefore the same procedure may have different costs based on the insurance company and the insurance policy the patient has. In addition, the amount may be different for non-insurers. In deciding how to prioritize radiology department upgrades or improvements, it could be useful to tie such improvements to anticipated revenue increases in a quantitative manner.

To this end, the disclosed systems and methods use a metric called a “billing rate” for a medical imaging procedure, which is defined as the ratio of the sum of the billing amounts for all occurrences of the procedure divided by the sum of the scan TAT for all occurrences of the procedure. That is, the billing rate for a procedure X, denoted BR_{X}, is given by:

$$B {R}_{X} = \frac{{\sum }_{\{ X \}} billing amount}{{\sum }_{\{ X \}} TAT} \qquad ( 1 )$$

where {X} denotes the set of occurrences of the procedure X in a time interval of operation of the medical department under analysis. In the illustrative examples for a radiology department, the scan TAT is used as the TAT; however, otherwise-defined turnaround times could be used. For example, the TAT could be a patient TAT defined as the interval between when the patient checks into the radiology department for the procedure and when the patient checks out. As another example, for a hematology laboratory a sample processing TAT could be defined as the interval between when the hematologist retrieves the blood sample and when the hematology report is filed. These are merely illustrative examples.

Equation (1) given above does not make any assumption that the billing amount per occurrence of the procedure is fixed, as the possibility of different billing amounts for different occurrences in the set {X} are captured by the summation in the numerator. On the other hand, if the billing amount is a fixed value for all occurrences of the procedure X in the set {X}, then the numerator of the billing rate BR_{X} expression can be expressed as a product as in Equation (2):

$$B {R}_{X} = \frac{N \cdot billing amount}{{\sum }_{\{ X \}} T A T} \qquad ( 2 )$$

where N denotes the number (i.e. count) of occurrences of procedure X in the set {X}. This expression is suitable if, for example, the medical department receives a fixed billing amount for each and every occurrence of the procedure X in the set {X}.

While the billing rate is a useful metric, it does not by itself provide a way to assess the impact of an optimization of the procedure.

Equation (1) or (2) is applicable to a set of empirical data, that is, to the set {X}. However, these expressions are not useful for performing “what if” scenarios to explore a hypothetical change in the medical procedure, since in this case no empirical data are available for the hypothetical change. To enable exploring hypothetical changes to the procedure, the statistical distribution of the TAT in the set {X} is modeled using a statistical distribution fitted to the TAT histogram of the empirical data set {X}. To permit efficient analysis of hypothetical changes, the statistical distribution is a parametric probability density function (PDF), and the impact of a hypothetical change in the procedure is represented by adjustment of a parameter (or multiple parameters) of parametric PDF representing (i.e. fitted to) the TAT distribution of the empirical data set {X}. The parametric PDF to be fitted is either chosen manually, e.g. by superimposing plots of different fitted parametric PDFs on the TAT histogram and receiving a manual selection, or by using a quantitative distance metric to programmatically identify the closest fitted parametric PDF.

With the TAT represented by a fitted probabilistic PDF, and assuming a fixed billing amount per occurrence of the procedure, the billing rate can be calculated using Equation (2), except that the TATs of the occurrences of the procedure in the empirical data set {X} are replaced by TATs for a number N_{draw} of hypothetical occurrences of the medical procedure drawn from the fitted parametric PDF with at least one parameter modified to represent a hypothetical change in the medical procedure. For a number N_{draw} of occurrences of the procedure X drawn from the fitted parametric PDF representing the empirical TAT statistical distribution but with the modified at least one parameter, the billing rate is (N_{draw}×billing amount) divided by the sum of the N_{draw} TATs drawn from the fitted parametric PDF representing the empirical TAT statistical distribution but with the modified at least one parameter. In other words, Equation (2) for the hypothetical situation can be expressed as follows:

$$B {R}_{X} = \frac{{N}_{draw} \cdot billing amount}{{\sum }_{\{ draw \}} T A T} \qquad ( 3 )$$

where {draws} represents the set of draws from the fitted parametric PDF, and as previously mentioned N_{draw} denotes the count of the draws.

In Equation (3), the billing amount is assumed to be fixed for all occurrences of the procedure. If this is not the case, then the analogous approach can be taken to handle the billing amount. The statistical distribution of the billing amount in the set {X} is modeled using a statistical distribution fitted to the billing amount histogram of the empirical data set {X}. The statistical distribution is again preferably a parametric PDF, and the impact of a hypothetical change in the procedure is represented by adjustment of a parameter (or multiple parameters) of the fitted parametric PDF representing the billing amount distribution. This leads to:

$$B {R}_{X} = \frac{{\sum }_{\{ draws \}} billing amount}{{\sum }_{\{ draw \}} TAT} \qquad ( 4 )$$

which corresponds to Equation (1) but now with the sum of the billing amounts for the occurrences being the sum of the billing amounts of the set {draws} drawn from the fitted parametric PDF representing the billing amount distribution.

It will also be appreciated that the billing rate computed for the data set {X} using Equation (1) (for variable billing amount) or Equation (2) (for fixed billing amount) can alternatively be closely approximated using respective Equation (4) or Equation (3) with the fitted parametric PDF representing the TAT (and the fitted parametric PDF representing the billing amounts in Equation (1)) being unmodified. For example, a close approximation to the billing rate given by Equation (2) is obtained by employing Equation (3) with no modifications to the fitted parametric PDF representing the TAT distribution of the empirical set {X}.

With this model, the impact on the billing rate of an optimization in the procedure can be estimated by (i) updating the TAT statistical distribution (i.e. fitted parametric PDF) to reflect the optimization (e.g. by modifying one or more parameters of the fitted PDF), and (ii) repeating the billing rate calculation using N_{draw} samples drawn from the updated TAT distribution. The update of the TAT distribution can be estimated based on the expected impact of the optimization. For example, if the TAT is a scan TAT and the optimization is a software upgrade that reduces scan time by 30%, then this information can be used to estimate the reduction in variance in the scan TATs.

While the following is described in terms of imaging procedures, the disclosed systems and methods recommend broadening the scope to medical procedures generally, with the TAT defined for the particular procedure. For example, a surgical procedure may have a TAT defined by the interval from when the patient is moved into the pre-op room to when the patient is moved into the post-op room.

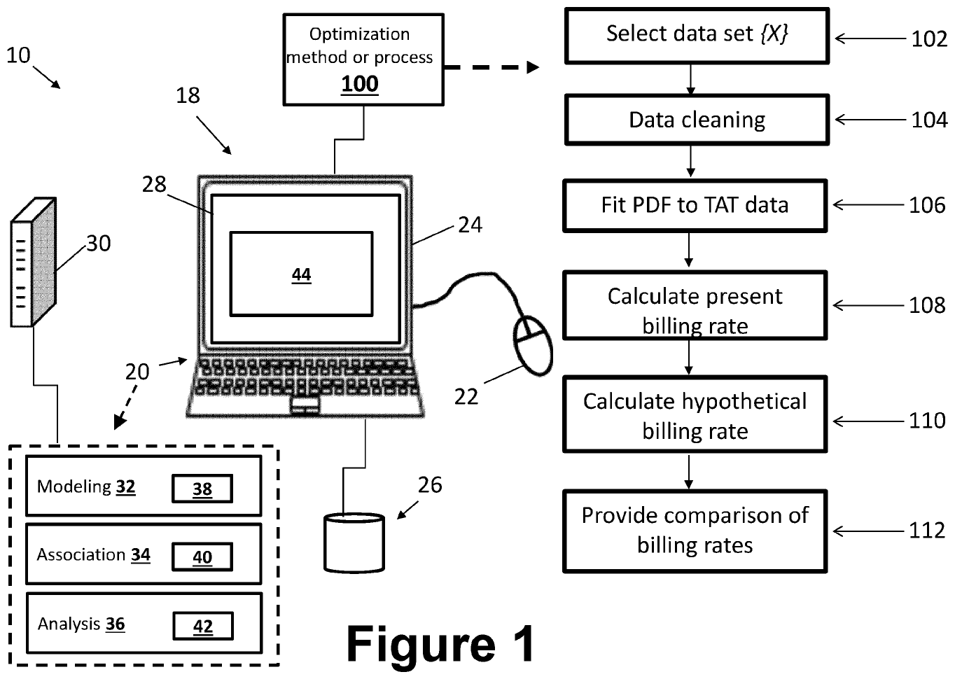

With reference to FIG. 1, an illustrative apparatus 10 for analyzing medical procedure billing rates is shown. As shown in FIG. 1, the apparatus 10 includes an electronic processing device 18, such as a workstation computer, or more generally a computer. The workstation 18 may also include a server computer or a plurality of server computers, e.g. interconnected to form a server cluster, cloud computing resource, or so forth, to perform more complex image processing or other complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The workstation 18 is in electronic communication with one or more databases 30, such as an Electronic Health Record (EHR) database storing information related to medical procedures performed at a medical facility. The information stored in the database 30 can include, for example, types of medical procedures, a number of occurrences for each occurrence of the medical procedures, an amount billed or reimbursed for each occurrence of the medical procedures, a turn-around-time (TAT) for each medical procedure, and so forth.

To analyze medical procedure billing rates at the medical facility, the at least one electronic processor 20 is programmed to implement one or more modules. As shown in FIG. 1, the at least one electronic processor 20 is programmed to implement a modelling module 32, an association module 34, and an analysis module 36. The modelling module 32 is configured to build one or more statistical models 38 for the TATs of the medical procedures, along with the amounted billed for each occurrence of the medical procedure (i.e., retrieved from the EHR database 30). The TATs (for example, an imaging scan TAT) can be treated as a univariate random variable and its distribution type and its parameter values can be identified. The billed or reimbursed amounts are usually fixed for a procedure type (for example, an X-Ray of Chest 1 view is billed at $212) and may vary only slightly due to other supplemental charges. Since there won't be much variability with the billing cost, it can be treated as a constant for a given procedure. The modelling module 32 is configured to clean the data retrieved from the EHR database 30. At times, certain radiology workflow events, like scan start time or end times, are not captured accurately, causing invalid data points. Such erroneous data points can be removed by consulting with subject matter experts to understand the range of acceptable values. Alternatively, statistical methods can be applied to remove data points outside the inter-quartile range as invalid (typically referred to as ‘outliers’).

The modelling module 32 is configured to assist a user of the workstation 18 identify a distribution type (i.e. a parametric PDF) that can be closely fitted to the retrieved TAT data. To this end, each candidate parametric PDF is fitted to the retrieved TAT data, e.g. using the Levenberg-Marquardt or other least squares optimization algorithm to optimize the parameters of the candidate parametric PDF to minimize the sum-of-squared differences (or other fit metric) between the fitted candidate parametric PDF and the TAT data. In some embodiments, the modeling module 32 is configured to generate visual representations of the retrieved empirical data and the fitted candidate parametric PDFs on some standard plots for inspection by the user, who can use the visual data for comparison and comparing it against fitted candidate parametric PDF distributions. Some examples of the provided visual data can include, for example, a Cullen and Frey graph, a histogram and probability density function (PDF) plot, an empirical and theoretical cumulative distribution function (CDF) plot (e.g., the theoretical distributions can be any distribution having a closest fit with the user hypothesis, such as a Burr or Lognormal distribution), a Quantile-Quantile (Q-Q) plot, a Percentile-Percentile (P-P) plot, among others. These are merely examples, and should not be construed as limiting. The user can examine the generated plots to determine a best fitting curve (e.g., based on a smoothness metric) or from another parameter (e.g., and area under the curve).

In other embodiments, this comparison can be quantitatively measured using a distance metric. For example, a Kolmogorov-Smirnoff (KS) distance can be measured to quantify a deviation of the CDF curve of empirical data with the CDF of each fitted candidate PDF. This quantitative metric can be used to programmatically decide the distribution type of the data. A lower KS value is indicative of a better fit of the empirical data to that distribution type.

Once the candidate fitted parametric PDF is chosen (thus from this point on being designated as the fitted parametric PDF for use in the subsequent operations), then the fitted parametric PDF and its corresponding CDF, constitutes the statistical model 38 of that data. The TAT for each procedure/protocol type is represented as the statistical model 38 and the billing/reimbursement amount is constant (or, alternatively, if the billing amount is not constant for all occurrences of the procedure then the model represents the billing amount by another fitted parametric PDF representing the billing amount, as discussed with reference to Equation (4)).

The association module 34 is configured to associate the TATs for a given medical procedure with an associated billing amount for that type of medical procedure. To do so, the association module 34 is configured to determine a present billing rate 40 for the medical procedure. The user can analyze the statistical model 38 (e.g., showing the TATs for the medical procedure) to determine a sum of TATs, and the (fixed) billing rate for that procedure. In one example, the present billing rate 40 can be calculated as the product of the fixed billing rate and the number of occurrence of the medical procedure to generate a product, which is divided by the sum of the TATs. In another example, the same product can be generated, and divided by a random sample of a number of occurrences of the medical procedure (denoted herein as the set {draws}) can be drawn from the PDF of the distribution type. This returns the frequency distribution of TAT (in minutes or another chosen time unit) which should match the actual data provided in the model 38.

The analysis module 36 is configured to analyze the model 38 and the present billing rate 40 to generate a hypothetical billing rate 42 for the medical procedure. To do so, the analysis module 36 is configured to perform what-if scenarios to generate the hypothetical billing rate 42. For example, the what-if scenarios can include changing the TATs for the medical procedure to determine the effect of that change on the present billing rate, which constitutes the hypothetical billing rate 42. A difference between the present billing rate 40 and the hypothetical billing rate 42 can be computed, and further supplemented with the volume of the medical procedure and savings in TATs to determine additional revenue the medical facility can expect for each of the medical procedure types. For example, a “X-Ray Chest” medical procedure can have a present billing rate 40 of $5.88 (based on 70 such procedures being performed in a day, at a rate of $212/exam). The user can decide to see what the effect of reducing the TAT for the X-Ray Chest procedure by 10% (e.g., by changing the workflow process, updating the hardware and software for this procedure, and so forth). The analysis module 36 can calculate the hypothetical billing rate 42 from this data. The hypothetical billing rate 42 can be multiplied by the difference in TAT of the model 38 to computed potential additional revenue. For example, a decrease in TAT by 10% for the X-Ray Chest procedure can result in a hypothetical billing rate 42, resulting in a determination of increasing revenue by $0.63/min for each procedure, and $44,730 for a quarter (e.g., 90 days).

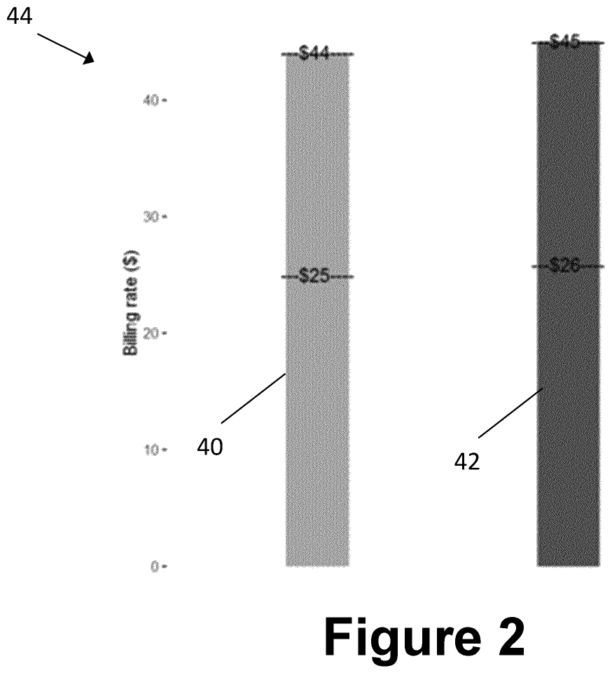

FIG. 2 shows an example visualization 44 showing a comparison of the present billing rate 40 and the hypothetical billing rate 42. The visualization 44 can be provided on the GUI 28 displayed on the display device 24. The visualization 44 shown in FIG. 2 is a bar graph with the “left” bar representing the present billing rate 40 and the “right” bar showing the hypothetical billing rate 42 for a specific procedure. The hypothetical billing rate 42 is generated from a what-if analysis of increasing revenue by $1/min, resulting in a new interquartile range is $26-$45 shown in the right bar, as compared to before which was $25-$44 in the left bar.

The apparatus 10 is configured as described above to perform medical procedure billing rate optimization method or process 100. The non-transitory storage medium 26 stores instructions which are readable and executable by the at least one electronic processor 20 of the workstation 18 to perform disclosed operations including performing the medical procedure billing rate optimization method or process 100. In some examples, the method 100 may be performed at least in part by cloud processing.

With continuing reference to FIG. 2 and with further reference to FIG. 1, an illustrative embodiment of the medical procedure billing rate optimization method 100 is diagrammatically shown as a flowchart in FIG. 1. In some embodiments, the medical procedure is a medical imaging procedure, and the TAT for the medical imaging procedure is a scan TAT running from when a first scan of the medical imaging procedure begins to when final clinical images are stored. The method 100 includes an operation 102 in which the data set {X} is retrieved from the storage 26. The data set {X} may, for example, consist of all occurrences of the procedure X performed in some chosen time interval (e.g. the last 3 months). Alternatively, the data set {X} may be some subset of those occurrences, e.g. all occurrences of an imaging procedure {X} performed by a specific imaging bay. In an optional operation 104, the data set {X} is cleaned, e.g. by removing any outliers. In an operation 106 performed by the modelling module 32. At the operation 106, a parametric PDF is fit to the data set of TATs for a medical procedure. The data set of TATs was retrieved from the EHR database 30 in the operation 102 and optionally cleaned in the operation 104, and the fitting of the PDF to the TAT dataset results in the generation of the model 38.

In some embodiments, the operation 106 includes fitting a plurality of different candidate parametric PDFs to the dataset of TATs for the medical procedure. A distance metric, such as the KS distance metric, can be calculated for each fitted candidate parametric PDF that measures similarity of the fitted candidate parametric PDF to the TATs of the dataset of TATs for the medical procedure. The fitted parametric PDF as the candidate parametric PDF with shortest calculated distance metric can be selected as the model 38.

In other embodiments, the operation 106 includes the fitting a plurality of different candidate parametric PDFs to the dataset of TATs for the medical procedure. The plurality of different fitted candidate parametric PDFs can be superimposed on a plot of the TATs of the dataset of TATs for the medical procedure. A manual selection of one of the plots of the different fitted candidate parametric PDFs is received via the at least one user input device 22. The fitted parametric PDF is the fitted candidate parametric PDF corresponding to the selected plot, constituting the model 38.

The method 100 also includes an operation 108 performed by the association module 34. At the operation 108, the present billing rate 40 is calculated or generated. To do so, a number of occurrences of the medical procedure can be drawn. The drawing of occurrences is selected, in some examples, from the data set of TATs (thereby applying Equation (2) for a fixed billing amount), or in other examples from the fitted parametric PDF (thereby applying Equation (3) for a fixed billing amount, with the unmodified fitted PDF). The present billing rate 40 represents an amount billed per minute (or other chosen unit time) of the scan interval (when using scan TATs) for the drawn occurrences of the medical procedure, and the TATs for the drawn occurrences of the medical procedure. The billing amount per procedure is assumed to be a constant amount billed per procedure for all drawn occurrences of the medical procedure in this example (so that Equation (2) or Equation (3) is suitable).

The method 100 further includes operations 110 and 112 performed by the analysis module 38. At the operation 110, the hypothetical billing rate 42 is generated or calculated with a hypothetical change. To do so, a number of hypothetical occurrences of the medical procedure is drawn from the fitted parametric PDF with at least one parameter modified to represent a hypothetical change in the medical procedure (it shall be noted that there does not exist a dataset of TATs for the hypothetical occurrences).

The hypothetical billing rate 42 is based on a hypothetical amount billed per procedure for the drawn hypothetical occurrences of the medical procedure, and the TATs for the drawn hypothetical occurrences of the medical procedure. The hypothetical amount billed per procedure is a constant hypothetical amount billed per procedure for all drawn hypothetical occurrences of the medical procedure. In some examples, the hypothetical billed amount 42 per medical procedure can be the same as the billed amount per procedure for the present case (i.e. for the data set {X}). In other examples, the fixed billed amount could be changed as part of the hypothetical, change (e.g. the user may be trying to answer the question: “Can we reduce our billed amount per procedure to a lower amount charged by a competitor by implementing the hypothetical change?”).

In further examples, the amount billed per procedure (either for present billing rate calculation 40 or the hypothetical billing rate calculation 42) could itself be a distribution in which case Equation (1) or Equation (4) with unmodified PDFs is used for the present billing rate calculation 40, and Equation (4) with modified PDFs is used for the hypothetical billing rate calculation 42. As discussed, in this variant a parametric PDF is also fitted to the dataset of billed amounts per procedure of the data set {X}. The dataset {X} here includes, for each instance of the procedure, both the TAT and the amount billed. The hypothetical billing rate 42 is then calculated based on a modification of a parameter of the billed amount PDF to represent the hypothetical change in the medical procedure (for example, if the hypothetical change involves a new tax of 5% added to the bill).

In some examples, the generating of the hypothetical billing rate 42 comprises generating a hypothetical product by multiplying the count of the drawn hypothetical occurrences of the medical procedure by the constant hypothetical amount billed per procedure and dividing the hypothetical product by a sum of the TATs for the drawn hypothetical occurrences of the medical procedure. That is, the hypothetical billing rate 42 is calculated according to Equation (3) for fixed billed amount or Equation (4) for variable billed amount.

At the operation 112, the visualization 44 of a comparison of the present billing rate 40 and the hypothetical billing rate 42 for the medical procedure with the hypothetical change can be provided on the GUI 28. The visualization 44 can be updated on the GUI 28 based on or more inputs from the user provided by the at least one user input device 22. For example, the drawing of a number of hypothetical occurrences and the generating of the hypothetical billing rate for the fitted parametric PDF can be repeated with different modifications of at least one parameter of the fitted parametric PDF representing corresponding different hypothetical changes in the medical procedure. That is, the user can select a parameter to be further modified, or a different parameter to be modified, to update the hypothetical occurrences/hypothetical billing rate 42. Different hypothetical billing rates 42 are generated corresponding to the different hypothetical changes in the medical procedure. The visualization 44 can then be updated with the different hypothetical billing rates 42 corresponding to the different hypothetical changes in the medical procedure for comparison with the present billing rates.

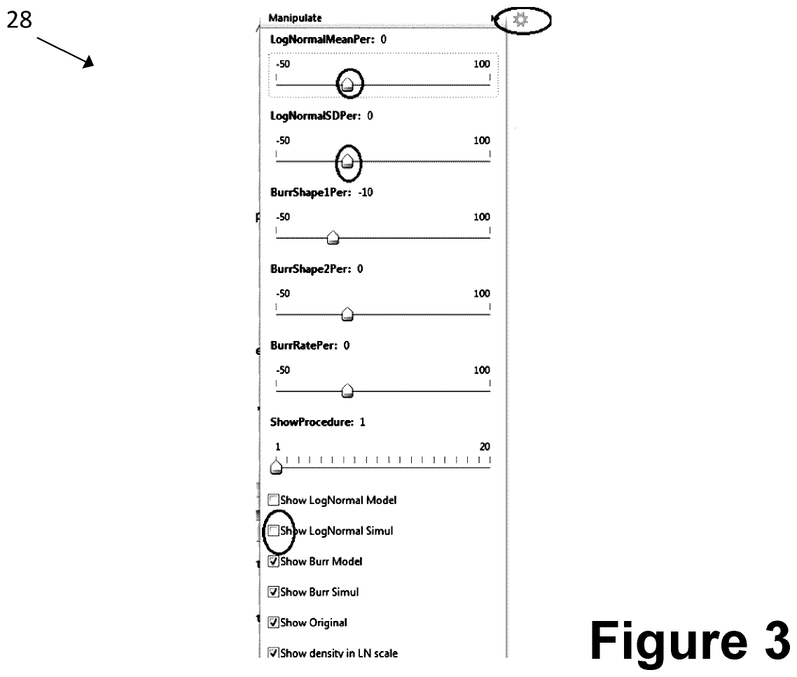

With reference back to FIG. 1 and with further reference to FIG. 3, in the operation 110 the parameter (or parameters) modification to the fitted parametric PDF representing the TAT can be input directly, for example using a GUI dialog implemented on the GUI 28 shown in FIG. 3 in which each parameter of the fitted parametric PDF is represented by a slider. In this approach, the user determines the appropriate change in parameter value or values based on knowledge of the expected impact on TATs of the hypothetical change in the medical procedure. For example, if the hypothetical change to an imaging procedure is an equipment and/or software upgrade to the medical imaging devices that is advertised by the imaging device manufacturer as reducing scan times for the procedure by 10%, then the expected change in the mean value of the parametric PDF should be reduced by 10%. If the mean value of the PDF is a parameter (as is the case, for example, in a Gaussian PDF), then the user suitably moves the slider for the mean parameter down by 10%. If the PDF does not have mean value as an explicit parameter, then the GUI dialog optionally still receives as input the 10% reduction in mean value of the PDF, and the GUI then computes adjustments to the parameters of the PDF such that the mean of the PDF is reduced by 10%. These are merely examples.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions readable and executable by at least one electronic processor to perform medical procedure billing rate optimization method, the method comprising:
fitting a parametric probability density function (PDF) to a dataset of turnaround times for a medical procedure;
drawing a number of occurrences of the medical procedure from the dataset of TATs or the fitted parametric PDF;
generating a present billing rate for the medical procedure based on an amount billed per procedure for the drawn occurrences of the medical procedure, and the TATs for the drawn occurrences of the medical procedure;
drawing a number of hypothetical occurrences of the medical procedure from the fitted parametric PDF with at least one parameter modified to represent a hypothetical change in the medical procedure;
generating a hypothetical billing rate for the medical procedure with the hypothetical change based on a hypothetical amount billed per procedure for the drawn hypothetical occurrences of the medical procedure, and the TATs for the drawn hypothetical occurrences of the medical procedure; and
providing a user interface (UI) displaying a visualization of a comparison of the present billing rate and the hypothetical billing rate for the medical procedure with the hypothetical change.

2. The non-transitory computer readable medium of claim 1, wherein the fitting of the parametric PDF to the dataset of TATs for the medical procedure includes:
fitting a plurality of different candidate parametric PDFs to the dataset of TATs for the medical procedure;
calculating a distance metric for each fitted candidate parametric PDF that measures similarity of the fitted candidate parametric PDF to the TATs of the dataset of TATs for the medical procedure; and
selecting the fitted parametric PDF as the candidate parametric PDF with shortest calculated distance metric.

3. The non-transitory computer readable medium of claim 2, wherein the distance metric comprises a Kolmogorov-Smirnoff distance metric.

4. The non-transitory computer readable medium of claim 2, wherein the fitting of the parametric PDF to the dataset of TATs for the medical procedure includes:
fitting a plurality of different candidate parametric PDFs to the dataset of TATs for the medical procedure;
superimposing the plurality of different fitted candidate parametric PDFs on a plot of the TATs of the dataset of TATs for the medical procedure; and
receiving, via at least one user input device, a selection of one of plots of the different fitted candidate parametric PDFs;
wherein the fitted parametric PDF is the fitted candidate parametric PDF corresponding to the selected plot.

5. The non-transitory computer readable medium of claim 1, wherein the method further comprises:
repeating the drawing of a number of hypothetical occurrences and the generating of the hypothetical billing rate for the fitted parametric PDF with different modifications of at least one parameter of the fitted parametric PDF representing corresponding different hypothetical changes in the medical procedure whereby different hypothetical billing rates are generated corresponding to the different hypothetical changes in the medical procedure;
wherein the providing of the UI includes displaying the visualization of a comparison of the present billing rate and the different hypothetical billing rates corresponding to the different hypothetical changes in the medical procedure.

6. The non-transitory computer readable medium of claim 1, wherein:
the amount billed per procedure is a constant amount billed per procedure for all drawn occurrences of the medical procedure; and
the hypothetical amount billed per procedure is a constant hypothetical amount billed per procedure for all drawn hypothetical occurrences of the medical procedure.

7. The non-transitory computer readable medium of claim 6, wherein:
the generating of the present billing rate comprises generating a product by multiplying the count of the drawn occurrences of the medical procedure by the constant amount billed per procedure and dividing the product by a sum of the TATs for the drawn occurrences of the medical procedure; and
the generating of the hypothetical billing rate comprises generating a hypothetical product by multiplying the count of the drawn hypothetical occurrences of the medical procedure by the constant hypothetical amount billed per procedure and dividing the hypothetical product by a sum of the TATs for the drawn hypothetical occurrences of the medical procedure.

8. The non-transitory computer readable medium of claim 6, wherein:
the generating of the present billing rate is calculated according to:
(A)(B)/(C)
where A comprises the count of the drawn occurrences of the medical procedure, B comprises the constant amount billed per procedure, and B comprises a sum of the TATs for the drawn occurrences of the medical procedure; and
the generating of the hypothetical billing rate comprises:
(X)(Y)/(Z)
where X comprises the count of the drawn hypothetical occurrences of the medical procedure, Y comprises the constant hypothetical amount billed per procedure, and Z comprises a sum of the TATs for the drawn hypothetical occurrences of the medical procedure.

9. The non-transitory computer readable medium of claim 1, wherein the medical procedure is a medical imaging procedure, and the TAT for the medical imaging procedure is a scan TAT running from when a first scan of the medical imaging procedure begins to when final clinical images are stored.

10. An apparatus, comprising:
a display device; and
at least one electronic processor programmed to:
generate a present billing rate for a medical procedure based on a number of occurrences of the medical procedure, an amount billed for the determined number of occurrences of the medical procedure, and a turn-around-time for the number of occurrences of the medical procedure;
generate a hypothetical billing rate based with at least one parameter modified to represent a hypothetical change in the medical procedure; and
provide a user interface on the display device displaying a visualization of a comparison between the present billing rate and the hypothetical billing rate.

11. The apparatus of claim 10, wherein the at least one electronic processor is further programmed to:
fit a parametric probability density function (PDF) to a dataset of turnaround times for a medical procedure; and
generate the present billing rate from the fit parametric PDF.

12. The apparatus of claim 11, wherein the fitting of the parametric PDF to the dataset of TATs for the medical procedure includes:
fitting a plurality of different candidate parametric PDFs to the dataset of TATs for the medical procedure;
calculating a distance metric for each fitted candidate parametric PDF that measures similarity of the fitted candidate parametric PDF to the TATs of the dataset of TATs for the medical procedure; and
selecting the fitted parametric PDF as the candidate parametric PDF with shortest calculated distance metric.

13. The apparatus of claim 12, wherein the distance metric comprises a Kolmogorov-Smirnoff distance metric.

14. The apparatus of claim 12, wherein the fitting of the parametric PDF to the dataset of TATs for the medical procedure includes:
fitting a plurality of different candidate parametric PDFs to the dataset of TATs for the medical procedure;
superimposing the plurality of different fitted candidate parametric PDFs on a plot of the TATs of the dataset of TATs for the medical procedure; and
receiving, via at least one user input device, a selection of one of plots of the different fitted candidate parametric PDFs;
wherein the fitted parametric PDF is the fitted candidate parametric PDF corresponding to the selected plot.

15. The apparatus of claim 10, wherein the at least one electronic processor is further programmed to:
repeat the generating of the hypothetical billing rate for the fitted parametric PDF with different modifications of at least one parameter of the fitted parametric PDF representing corresponding different hypothetical changes in the medical procedure whereby different hypothetical billing rates are generated corresponding to the different hypothetical changes in the medical procedure; and
update the visualization corresponding to the different hypothetical changes in the medical procedure.

16. The apparatus of claim 10, wherein:
the amount billed per procedure is a constant amount billed per procedure for all occurrences of the medical procedure; and
the hypothetical amount billed per procedure is a constant hypothetical amount billed per procedure for all hypothetical occurrences of the medical procedure.

17. The apparatus of claim 16, wherein the at least one electronic processor is further programmed to:
generate the present billing rate by generating a product by multiplying the count of the occurrences of the medical procedure by the constant amount billed per procedure and dividing the product by a sum of the TATs for the occurrences of the medical procedure; and
generate the hypothetical billing rate by generating a hypothetical product by multiplying the count of the hypothetical occurrences of the medical procedure by the constant hypothetical amount billed per procedure and dividing the hypothetical product by a sum of the TATs for the hypothetical occurrences of the medical procedure.

18. The apparatus of claim 16, wherein the at least one electronic processor is further programmed to:
generate of the present billing rate according to:
(A)(B)/(C)
where A comprises the count of the occurrences of the medical procedure, B comprises the constant amount billed per procedure, and B comprises a sum of the TATs for the occurrences of the medical procedure; and
generate the hypothetical billing rate according to:
(X)(Y)/(Z)
where X comprises the count of the hypothetical occurrences of the medical procedure, Y comprises the constant hypothetical amount billed per procedure, and Z comprises a sum of the TATs for the hypothetical occurrences of the medical procedure.

19. The apparatus of claim 1, wherein the medical procedure is a medical imaging procedure, and the TAT for the medical imaging procedure is a scan TAT running from when a first scan of the medical imaging procedure begins to when final clinical images are stored.

20. A medical procedure billing rate optimization method, comprising:
fitting a parametric probability density function (PDF) to a dataset of turnaround times for a medical procedure;
generating a present billing rate for the medical procedure based on an amount billed per procedure for occurrences of the medical procedure drawn from the dataset of TATs for the medical procedure, and the TATs for the drawn occurrences of the medical procedure;
generating a hypothetical billing rate for the medical procedure with the hypothetical change based on a hypothetical amount billed per procedure for hypothetical occurrences of the medical procedure drawn from the fitted parametric PDF with at least one parameter modified to represent a hypothetical change in the medical procedure, and the TATs for the drawn hypothetical occurrences of the medical procedure;
providing a user interface (UI) displaying a visualization of a comparison of the present billing rate and the hypothetical billing rate for the medical procedure with the hypothetical change;
generating different hypothetical billing rates for the fitted parametric PDF with different modifications of at least one parameter of the fitted parametric PDF representing corresponding different hypothetical changes in the medical procedure; and
updating the visualization of the comparison of the present billing rate and the different hypothetical billing rates corresponding to the different hypothetical changes in the medical procedure.
